(19) United States
(12) Patent Application Publication
(10) Pub. No.: US 2023/0383239 A1
(43) Pub. Date: Nov. 30, 2023

(54) MICROSCALE CELL FILTER

(71) Applicant: INL - INTERNATIONAL IBERIAN NANOTECHNOLOGY LABORATORY, Braga (PT)

(72) Inventors: Lorena DIÉGUEZ, Braga (PT); Silvina SAMY, Braga (PT); Marta OLIVEIRA, Braga (PT); João GASPAR, Braga (PT)

(21) Appl. No.: 18/366,467

(22) Filed: Aug. 7, 2023

Related U.S. Application Data
(63) Continuation-in-part of application No. 16/349,656, filed on May 14, 2019.

(51) Int. Cl. C12M 1/00; C12M 1/12

(52) U.S. Cl. CPC C12M 47/04; C12M 23/06; C12M 23/20

(57) ABSTRACT

A microscale cell filter for separating and isolating cancer cells within a sample having an inlet flow channel; an outlet flow channel; and a single row of a plurality of post elements between the inlet flow channel and the outlet flow channel, the plurality of post elements being interspaced, forming a plurality of gaps, each gap formed in between two adjacent post elements; the plurality of post elements is arranged such that a sample flowing from the inlet flow channel to the outlet flow channel passes through the plurality of gaps; the plurality of post elements is arranged such that a width of each gaps is 3 to 8 micrometers; and each gap has an aspect ratio between its height and width in the range of 3.5 to 5, thereby trapping the cells within the sample at an upstream side of the single row of a plurality of post elements.

### CROSS-REFERENCE TO RELATED APPLICATIONS

This application is a continuation in part application of U.S. application Ser. No. 16/349,656, filed May 14, 2019, which in turn is a 371 national stage entry of International Application No. PCT/EP2016/078406, filed on Nov. 22, 2016, the contents of each of which are incorporated herein by reference.

### TECHNICAL FIELD

The present invention relates to a method for separating and isolating cancer cells within a sample including, but not limited to, circulating tumor cells within a blood sample.

### BACKGROUND

Metastasis is an underlying cause of morbidity and cancer-related mortality worldwide. Circulating tumor Cells, CTCs, may be deposited from a primary tumor and disseminated through the blood stream to other parts of the body, including potentially invading other organs and causing metastasis thereof. CTCs are not homogeneous and comprise different sizes and phenotypes. The detection of CTCs may provide valuable information for the clinical management of cancer patients since they provide a real-time snapshot of the current tumor burden in the body of the patient. However, the CTCs are, when available at all, present in extremely low concentrations, such as 1 to 10 CTCs per billion blood cells. Isolating the CTCs is therefore problematic.

Several methods for isolating and characterizing CTCs are available but every method leave much to be desired in terms of cost, time, effectiveness, simplicity to the user, etc. Most systems of enriching and characterising CTCs rely on the expression of the epithelial cell adhesion molecule (EpCAM) and/or on immunofluorescence analysis with antibodies targeting cytokeratins (CKs). Evidence suggests that CTCs can, for example, undergo Epithelial Mesenchymal Transition (EMT), thereby presenting a variety of sub-populations with dissimilar phenotypical and functional characteristics, leading to poorer prognosis and misleading results. There is thus a need for an analytical system capable of more efficient handling samples with sub-populations of cells.

### SUMMARY

In view of the above, it is an object of the present invention to provide a microscale cell filter for trapping a sub-portion of cells within a sample. It is further an object of the present invention to overcome the obstacles presented by the prior art. These and other objects are at least partly met by the invention as defined in the independent claim. Preferred embodiments are set out in the dependent claims.

According to a first aspect, a microscale cell filter for trapping a sub-portion of cells within a sample is provided. The cell filter comprises: an inlet flow channel; an outlet flow channel; and a plurality of post elements arranged in a single row between the inlet flow channel and the outlet flow channel, also arranged perpendicular to the direction of the flow, wherein the plurality of post elements is interspaced, thereby forming a plurality of gaps, each gap being formed in between two adjacent post elements; wherein the plurality of post elements is arranged such that a flow of the sample flowing from the inlet flow channel to the outlet flow channel passes through the plurality of gaps; wherein the plurality of post elements is arranged such that a width of each gaps is 3 to 8 micrometers, in one embodiment, the width of each gap is 3 to 6 micrometers; and wherein each of the plurality of post elements has an elongation such that each gap has an aspect ratio between its width and its height being larger than 3.5, preferably in the range of 3.5 to 5, thereby trapping cells of the sample at an upstream side of the plurality of post elements.

By providing a cell filter that physically separates and isolates cells, enabling their enumeration and characterization, a real-time snapshot of current tumor burden is enabled. The low invasive technique according to the present invention filters cells by size and deformability. The separation and isolation are thereby not dependent on any staining or antibodies required by most methods according to prior art. The cells trapped on the filter may be extracted for subsequent analysis. The microscale cell filter may be comprised on a microfluidic chip. In the latter case, the trapped cells may be analyzed directly inside the microfluidic chip.

Cells, e.g. CTCs, that are larger than the gaps and are substantially rigid may be trapped in the filter. For example, if a rigid cell has a diameter of 8 micrometers and the gap is between 3 to 8 micrometers, the cell may be trapped in the filter. In one embodiment, the width of each gap is 3 to 6 micrometers. To obtain the same effect in flexible and in rigid materials that make the microfluidic filter, the gap width of the rigid materials needs to be adjusted to the effective dimensions when the flexible system is under pressure (flow conditions), this the preferred gap diameter is between 3 to 8 micrometers.

As there is a plurality of gaps in the cell filter and/or since the gaps have the above mentioned aspect ratio, the flow may continue to flow from the inlet flow channel to the outlet flow channel without significantly affecting the flow pressure of the device.

In the case of cells being larger than a gap, but deformable, the cells may squeeze through the gap from the inlet flow channel to the outlet flow channel using the elongation of the gap. This is the case for some cells in blood. For example, white blood cells can have diameters up to 20 micrometers but, due to their small or lobulated nucleus, may be deformed in the z-axis by the pressure from the flow against the post elements which enables the cells to pass through.

Cells that are smaller than the gap may pass freely through the filter. This is the case of red blood cells, which are the majority of cells in whole blood, and have a disc shape with an average diameter of 5 microns.

As the number of components in the filter is low, possibly comprising only post elements and channels, easy and cheap manufacturing is enabled. Further, the cell filter may be manufactured as a monolayer device, that does not include different layered structures of different heights, which does make the fabrication easy. The cell filter also enables an easy-to-use protocol, thereby lowering the need for high-skilled personnel.

Furthermore, since single cell capturing is possible with the filter, small concentrations of a sub-portions of the sample may be identified. As a non-limiting example, circulating tumor cells (CTCs) may be present in blood in very low concentrations, such as 1-10 cells per billion blood cells. The CTCs have diameters above 5 micrometers and generally higher density and lower compressibility than other cells. The cell filter may therefore be used to detect CTCs in blood by letting small and/or deformable cells to pass through the gaps while trapping the CTCs in the filter, enabling their analysis for accurate prognosis, personalized treatment and therapy monitoring.

Whole blood samples that run through the microscale cell filter do not need any pretreatment. The trapped cells may be analyzed in the cell filter (in situ). This may e.g. be made using a microscope. Alternatively or in combination, the trapped cells may be extracted from the cell filter and later analyzed. Moreover, since the present invention does not need any pre-treatment the trapped cells are viable. Hence, extracted cells may be cultured and/or proliferated for further analysis. Further, the decision to which analyzing method to use may be taken based on real-time information obtained during the filtering. For example, if the number of trapped cells exceeds a certain number, the analysis may be made in-situ, while if the number of trapped cells is less than a certain number, the cells might be extracted and cultured for more accurate results.

Moreover, urine samples may be run through the cell filter.

Hence, the present microscale cell filter allows for physical sorting, e.g. the microscale cell filter is not dependent on staining and antibodies, or on phenotypical characteristics. The microscale cell filter is filtering by size. The microscale cell filter allows for filtering by deformability. Since CTCs have a large diameter, as compared with many other cells contained in body fluids, and are more rigid compared to most other cells contained in body fluids, they will be trapped by the present cell filter while other cells are able to flow through the cell filter. Hence, the microscale cell filter may be used for isolation of CTCs. The microscale cell filter allows for trapping of all CTCs regardless of their phenotype, meaning that both epithelial and also mesenchymal CTCs are trapped. The microscale cell filter may be used for taking a real-time snapshot of current tumor burden. The microscale cell filter may be used for liquid biopsy of whole blood, urine and other body fluids. For example, the microscale cell filter allows for direct processing of whole blood, with no need of sample preparation or pretreatment. The microscale cell filter is cheap and easy to produce. The microscale cell filter may be easily integrated with a biosensor for fast and cost-effective in-situ tumor cell phenotypic and molecular profiling.

The term ‘microscale’ should be construed as a filter that has at least one of its dimensions, for example height or width of the flow channel in the microscale size. A suitable platform format would be to use a microfluidic chip. The microfluidic chip may be manufactured from, comprise, or essentially consist of a material selected from the non-exhaustive list consisting of silicon, glass, paper, and polymers, such as SU-8, PDMS, PC, PET, PE/PET, polyimide, and PMMA, or any combinations thereof.

Some parts of the microscale cell filter may be larger than on the microscale. Furthermore, the microscale is not limited to above one micrometer. Some of the dimensions of the microscale cell filter may therefore be under one micrometer, such as 0.1 micrometer or 0.01 micrometer.

A filter may be a hindering portion of the cell filter. The effects of the filter may be one of the non-limiting examples of stopping cells from flowing through or collecting cells in the filter. Furthermore, the term ‘trapping’ should be construed in the same broad sense in that the trapping may be performed by capturing cells or construed as only blocking cells from passing through.

As the plurality of post elements are arranged between the inlet flow channel and the outlet flow channel. The word ‘between’ should in the purpose of this application be construed as “on a border connecting the inlet flow channel with the outlet flow channel”.

The interspacing of the plurality of post elements comprises the space, i.e. the gap between two post elements in the plurality of post elements. The upstream side is on the side of the inlet flow channel. However, if the flow in the channel is reversed, with liquid flowing from the outlet flow channel to the inlet flow channel, the upstream side is still, for the clarity of this application, the side of the inlet flow channel.

Each of the plurality of post elements may be cylindrically shaped. Having cylindrical post elements make manufacturing easier and the trapping effect easier to control due to the predictability of the structural geometry of the post elements. Furthermore, if the shapes of the post elements are cylindrical, the contact surface of in the longitudinal direction of the flow in the flow channels may be reduced. This may reduce the risk of cells adhering to the surface of the post elements and ensures proper flow pressure.

According to a further embodiment, the cylindrical shape is a circular cylinder. By having circular cylindrical shapes of the post elements the compressing effect on deformable cells may be enhanced. Making it more easy for large deformable cells to go through the microscale cell filter. Moreover, two adjacent post elements having circular cylindrical shape guides the cells into the gap between said two post elements in an efficient manner. Hence, the two features of compressing and guiding may thus act together, thereby enhancing deformation filtering.

The height of the plurality of post elements may be identical.

The microscale cell filter may further comprise a substrate, wherein the plurality of post elements is integrally formed with the first substrate. By producing the post elements identical and integral with the substrate, cost effectiveness and manufacturing simplicity may be achieved.

The substrate and the plurality of post elements may be formed by a PDMS layer. PDMS is biocompatible and ensures viability of the cells.

The cell filter may further comprise a cover, wherein the plurality of post elements is arranged in between the substrate and the cover.

The cover may be transparent. Transparency allows for optical imaging as well as visually observing the status of the microscale cell filter.

The surfaces of the plurality of post elements may comprise a surfactant. The surfactant may be provided by contacting the post elements with a pluronic acid. Also the inner surface of the substrate and/or the cover may comprise a surfactant. The surfactant may provide smooth transitioning of the cells from the inlet flow channel to the outlet flow channel. Furthermore, as the cells within the present cell filter may maintain their viability, surface treatment with pluronic acid efficiently prevents adhesion of cells. This will minimize isolation of unwanted cells such as white blood cells. Further it will simplify harvesting of trapped cells upon flow reversal through the cell filter.

A width of each post element may be 15 to 40 micrometers, preferably 25±10% micrometers.

A further scope of applicability of the present invention will become apparent from the detailed description given below. However, it should be understood that the detailed description and specific examples, while indicating preferred embodiments of the invention, are given by way of illustration only, since various changes and modifications within the scope of the invention will become apparent to those skilled in the art from this detailed description.

Hence, it is to be understood that this invention is not limited to the particular component parts of the device described or steps of the methods described as such device and method may vary. It is also to be understood that the terminology used herein is for purpose of describing particular embodiments only, and is not intended to be limiting. It must be noted that, as used in the specification and the appended claim, the articles “a,” “an,” “the,” and “said” are intended to mean that there are one or more of the elements unless the context clearly dictates otherwise. Thus, for example, reference to “a unit” or “the unit” may include several devices, and the like. Furthermore, the words “comprising”, “including”, “containing” and similar wordings does not exclude other elements or steps.

### BRIEF DESCRIPTIONS OF THE DRAWINGS

The above and other aspects of the present invention will now be described in more detail, with reference to appended drawings showing embodiments of the invention. The figures should not be considered limiting the invention to the specific embodiment; instead they are used for explaining and understanding the invention.

As illustrated in the figures, the sizes of layers and regions are exaggerated for illustrative purposes and, thus, are provided to illustrate the general structures of embodiments of the present invention. Like reference numerals refer to like elements throughout.

FIGS. 1a and 1b illustrates schematic top views of a microscale cell filter.

FIG. 2 illustrates a cross-sectional view of the cell filter, taken along the line A-A in FIG. 1b.

FIG. 3 illustrates a schematic perspective view of a microscale cell filter.

FIG. 4 illustrates a schematic top view of an alternative embodiment of a microscale cell filter.

### DETAILED DESCRIPTION

The present invention will now be described more fully hereinafter with reference to the accompanying drawings, in which currently preferred embodiments of the invention are shown. This invention may, however, be embodied in many different forms and should not be construed as limited to the embodiments set forth herein; rather, these embodiments are provided for thoroughness and completeness, and to fully convey the scope of the invention to the skilled person.

A sample comprising biological cells may flow from an inlet to an outlet while passing a filter section formed by a plurality of interspaced post elements. Depending on the size and compressibility of the passing cells, some cells may be trapped in the gaps between the post elements. As the size and compressibility is a characteristic of the cell type, desired type of cells may be trapped in the cell filter, without any need for chemical targeting or fixation.

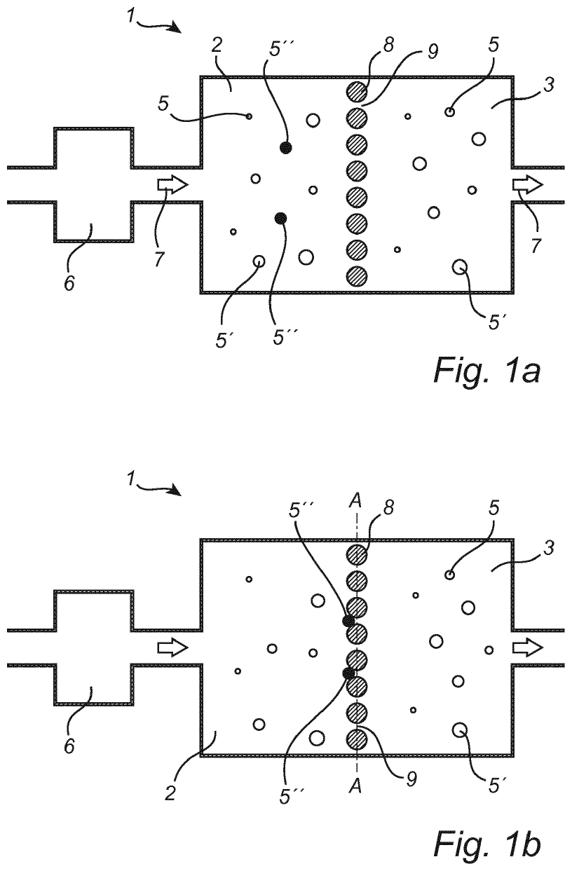

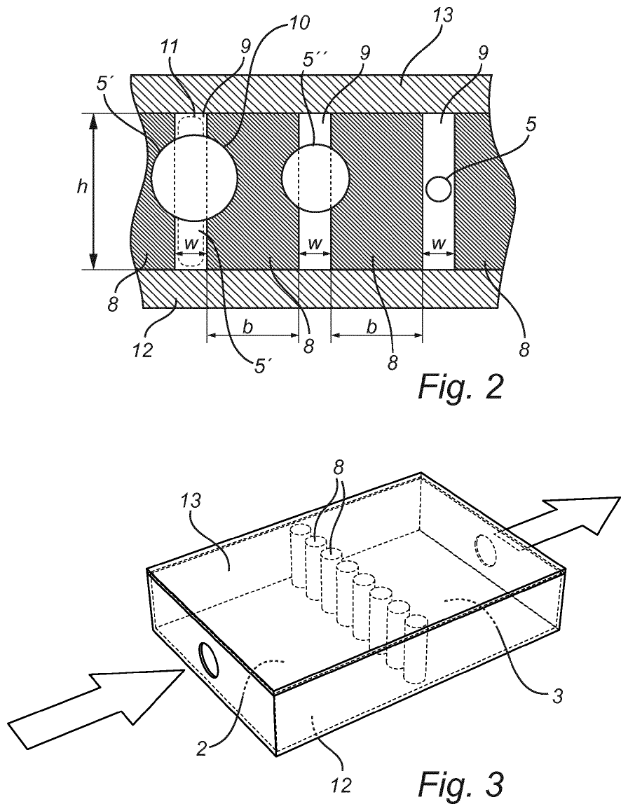

With reference to FIGS. 1a, 1b and 2, a microscale cell filter 1 for trapping a sub-portion of cells within a sample will now be described. The cell filter 1 comprises an inlet flow channel 2 and an outlet flow channel 3. The inlet flow channel 2 is configured to receive a sample. The sample is a liquid. The sample may for example be whole blood. However, other samples of body fluid, e.g. urine may also be used with the present microscale cell filter. The sample comprises cells 5, 5′, 5″. In the in FIG. 1 shown embodiment the sample comprises three different type of cells 5, 5′, 5″, namely relatively small cells, relatively large deformable cells 5′ and circulating tumor cells, CTCs, 5″. However, the sample may comprise more or less type of cells.

The microscale cell filter 1 may be arranged on a microfluidic chip (not shown). For the purpose of this application however, the cell filter 1 will be described in the context of a microfluidic chip. The flow channels 2, 3 of the microscale cell filter 1 may have a cross sectional area taken perpendicular to a flow direction of the sample through the cell filter 1 of 25 to 100 000 μm². The length of the flow channels 2, 3 may be, for example, 0.1 to 100 cm.

The cell filter 1 may be created using standard manufacturing techniques. The cell filter 1 may be made by using soft lithography. The flow channels 2, 3 may have various suitable shapes and geometries. For example, the geometry perpendicular to the flowing of the sample may be rectangular, oval or circular shaped. The flow channels 2, 3 may further have a serpentine or undulating elongation thereby allowing for a compact design of the microfluidic chip.

The microscale cell filter 1 may comprise a flow generator 6, configured to provide a flow of the sample through the cell filter 1. The flow generator 6 may be directly or indirectly connected to the inlet flow channel 2. For example, the flow generator 6 may be indirectly connected by means of one or more tubings or tubes, channels or capillaries, or combinations thereof. The flow generator 6 may provide a flow such as a peristaltic flow, a continuous or a periodical flow, or combinations thereof. The flow may be provided at different flow rates. The flow may be turned on and off during different time intervals. The flow generator 6 may be a pump, such as a syringe pump, a peristaltic pump or a pressure pump. The flow generator 6 may be operated manually or energized.

The direction of the flow through the cell filter 1 is schematically illustrated by arrows 7.

The cell filter 1 further comprises a plurality of post elements 8, the plurality of post elements 8 being arranged between the inlet flow channel 2 and the outlet flow channel 3. The plurality of post elements 8 may be parallel to each other and perpendicular to the direction of the flow. The plurality of post elements 8 is interspaced, thereby forming a plurality of gaps 9. The gaps 9 are formed between adjacent post elements 8.

The sample that passes through the cell filter 1 passes the plurality of gaps 9. There might be a different amount of sample flowing through different parts of the cell filter 1, depending on the chosen dimensions. For example, since the flow velocity in a microfluidic channel is different in the middle of the channel compared too close to the walls, a higher amount of sample may flow in the middle of the channel. Furthermore, the frequency of gaps 9 may be different in different parts of the cell filter 1. For example, the frequency of gaps 9 may be higher in the center of the flow channel than close to the walls.

The plurality of post elements 8 are identical to each other.

With reference to FIG. 2, the plurality of post elements 8 may be arranged such that a width w of each gap is 3 to 8 micrometers. In one embodiment, the width w of each gap is 3 to 6 micrometers. In a preferred embodiment, the width w of the gaps 9 are 5±10% micrometers.

Furthermore, the plurality of post elements 8 may comprise an elongation h such that each gap 9 comprises an aspect ratio between the width w and the elongation h of the gap being more than 3.5. In a preferred embodiment the aspect ratio is in the range of 3.5 to 5. In an even more preferred embodiment the aspect ratio is 4±10%. The gaps 9 may have the cross-sectional dimensions of a width W of 5±10% micrometers by a height h 20±10% micrometers. A gap 9 comprising the dimensions explained above may be arranged to trap a sub-portion of cells 5, 5′, 5″ carried in the sample.

The cross section of the post elements 8, illustrated a post element width b, may be in the range of 15 to 40 micrometer, preferably 25±10% micrometer. Hence, in case of circular cylindrical post elements 8 the diameter of the post elements 8 may be in the range of 15 to 40 micrometer, preferably 25±10% micrometer. Hence, the width b of the post elements 8 are preferably larger than the width W of the gaps 9.

There are different mechanisms acting in conjunction to trap the sub-portion of cells 5, 5′, 5″ carried in the sample in the gaps 9. For the facilitation of reading, each mechanism will be explained separately but in practice, a combination of the mechanisms is in play.

The cell filter 1 may filter out a sub-portion of cells 5, 5′, 5″ from the sample 4 based on size. For example, a cell having a diameter that is wider than the width w of the gaps 9, e.g. wider than the preferred width w of 5±10% micrometers, may not enter through the gaps 9. This since such cells may be physically stopped by the plurality of post elements 8. An example of cells that are normally wider than the width w of the gaps 9 are CTSs CTCs are around 10-20 micrometers, or more, in diameter and have a relatively rigid body, due to the size of their nucleus. Hence, CTCs do not deform to great extent and are hence trapped in the cell filter 1. This is illustrated in FIGS. 1b and 1n the middle of FIG. 2.

In the case that the trapped cell does not cover the whole gap 9, the flow of the sample may continue to flow through the gap 9.

Cells 5 having a smaller diameter than the width w of the gap 9 pass through the cell filter 1. Hence, such cells 5 are freely moving from the inlet flow channel 2 to the outer flow channel 3. This is illustrated in FIG. 1 and in the right hand side of FIG. 2. In one embodiment, whole blood is filtered, in which many cells have a diameter equal or smaller than 5 micrometers, such as red blood cells (5 micrometers) and platelets (2 to 3 micrometers). A cell having a diameter smaller than the width w of the gap 9 passes through the cell filter 1, regardless of its other properties, such as deformability and/or density.

Biological cells may be deformable. The cytoplasm of the cell may be more deformable than the nucleus of the cell. As illustrated in the left hand side of FIG. 2 a cell having larger diameter than the width w of the gap 9 is hindered by the plurality of post elements 8, the cell 5′ in its initial non-deformed state is in FIG. 2 illustrated as having the cross section indicated by the solid line 10. In FIG. 2. a deformable cell 5′, in its non-deformed state as illustrated by the solid line 10 cross section, is about to encounter the plurality of post elements 8. However, since the cell 5′ is deformable, the deformable cell 5′ may deform along the z-axis and pass the cell filter 1 through the gap 9. The cell 5′ in its deformed state is in FIG. 2 illustrated as having the cross section indicated by the dotted line 11. An example of a deformable type of cell 5′ is, neutrophils constituting 60 to 70 percent of white blood cells. Neutrophils typically have a diameter of 14 micrometers. The cross-sectional area of the neutrophil is thus approximately 150 μm². if the width w of a given gap 9 in which neutrophils is about to pass through is 5 micrometers, the height h of the gap 9 must at least be around 30 micrometers. However, the neutrophils may also deform in the longitudinal direction letting the deformable neutrophils pass through a gap of 5 times 20 micrometers. Hence, a longitudinal elongation of the deformable cell 5′ may lower the required cross sectional area of the gap 9 in order for the deformable cell 5′ to pass the cell filter 1. As deformable cells 5′ may pass through the cell filter 1 as long as there is enough room for them to squeeze through, the upper limits of the dimensions of the gaps 9 may set by dimension requirements.

The filtered sample that have passed through the cell filter 1 may be discarded. Alternatively, the filtered sample may be analyzed further.

Analysis of the trapped cells 5″ may be performed while the isolated cells are situated in the cell filter 1. As a non-limiting example, the trapped cell may be counted using an optical microscope. In connection with analysis, the trapped cells 5″ may also be stained for easier visualization. The trapped cells 5″ may for example be stained by targeting the sub-portion of cells with antibodies and subsequently marking the antibodies with fluorescent molecules, such as cytokeratin and vimentin. A person skilled in the art realizes that there are many different methods for cellular analysis available. The trapped cells 5″ may also be extracted and examined outside of the cell filter 1. For example, as the trapped cells 5″ are still viable, the cells 5″ may be cultured in growth medium and proliferated for more convenient analysis. The trapped cells 5″ may also be lysed to recover their nucleic acids content for molecular analysis The trapped cells 5″ may be extracted by reversing the flow such that the flow flows from the outlet flow channel 3 to the inlet flow channel 2 thereby releasing the trapped cells 5″ from the post elements 8.

Now referring to FIG. 3, the post elements 8 may be of cylindrical geometry such that each post element 8 comprise a constant cross-sectional shape along the entire height the post element 8. Furthermore, the cylindrical shape may be a circular cylinder. A cylindrical shape of the post elements 8 may facilitate the guidance of the cells into the gaps 9. Further, by having circular cylindrical post elements 8, the cells that are either deformable enough to pass through, given that the dimensions of the gap 9 is such that the cell is allowed passage, or small enough to not be hindered by the plurality of post elements 8 will not adhere to the walls of the post elements 8. The longitudinal friction of cells against the post elements 8 is thereby reduced. Hence, clogging may be reduced.

To reduce friction against the post elements 8 of the cells 5, 5′ passing through the cell filter, a surfactant may be placed on the post elements 8. A non-limiting example of surfactant is pluronic acid. Further examples of surfactant are tween, sodium desoxycholate, SDS. The surfactant may further facilitate the releasing of the trapped cells 5″ upon extraction.

The cell filter 1 may have any suitable shape or form, for example a flat and thin shape. The cell filter 1, and its elements, may be manufactured from, essentially consist of, or comprise a material selected from the list consisting of silicon, glass, paper, and polymers, such as, but not limited to, PDMS, PC, PET, PE/PET, polyimide, and PMMA, and combinations thereof.

Again, with reference to FIG. 3, the cell filter 1 comprises a substrate 12. The post elements 8 may be arranged on the substrate 12. The plurality of post elements 8 may be integrally formed with the substrate 12. The substrate 12 may be made of a polymer material such as polydimethylsiloxane, PDMS. The post elements 8 may be made of a polymer material such as polydimethylsiloxane, PDMS. It is however, realized that the substrate 12 and/or the post elements 8 may be manufactured from, essentially consist of, or comprise a material selected from the list consisting of silicon, glass, paper, and polymers, such as PDMS, PC, PET, PE/PET, polyimide, and PMMA, and combinations thereof. The cell filter 1 further comprises a cover 13. The cover 13 may be transparent. As a non-limiting example, the cover 13 may be made of glass. A transparent cover 13 may facilitate on-chip analysis.

An example of manufacturing method for the microscale cell filter 1 may be to have the plurality of post elements 8 be integrally formed with the substrate 12. The substrate 12 may form part of a flow channel for the sample. The substrate 12 may furthermore be molded in PDMS at the same time as the molding of the plurality of post elements 8.

It is realized that other modules may be connected to the cell filter 1 such as other microfluidic components. For example, any of the following: DNA extraction with reagent kits, sample preparation modules, CTC isolation and lysis modules, ctDNA separation, mixing module and biosensors enabling fast and cost-effective in-situ tumor cell phenotypic and molecular profiling. Multiple cell filters 1 may also be connected in parallel to process more sample.

Below a non-limiting example of a manufacturing process of a microscale cell filter 1 will be discussed. Deep Reactive Ion Etching (DRIE) may be used to etch a microstructure into a silicon wafer. Each cycle of etching may be followed by a deposition of Teflon-like polymer on the walls to ensure unidirectional etching. The fabrication of a polymeric replica of the microstructure may be done by a molding procedure, in which the microstructures are placed on a silicon wafer and PDMS is poured on top. The silicon wafer, including the silicon microstructures may be removed and a PDMS with a negative of the microstructures may be provided. Glass may be bonded to the PDMS using oxygen plasma treatment.

The person skilled in the art realizes that the present invention by no means is limited to the preferred embodiments described above. On the contrary, many modifications and variations are possible within the scope of the appended claims.

Furthermore, many other body fluids may be analyzed.

Further, the number of inlet flow channels 2 may vary. The microscale cell filter 1 may comprise one single inlet flow channel 2. The microscale cell filter 1 may comprise a plurality of inlet flow channels 2.

Moreover, the number of outlet flow channels 3 may vary. The microscale cell filter 1 may comprise one single outlet flow channel 3. The microscale cell filter 1 may comprise a plurality of outlet flow channels 3.

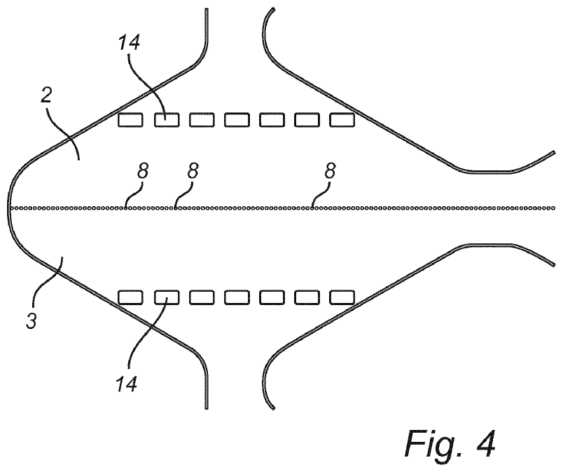

Further, as illustrated in FIG. 4 the inlet flow channel 2 may comprise a further filtering stage comprising a plurality of further post elements 14. The further post elements 14 may be formed by rectangular-shaped posts of 200 micrometer×100 micrometer. It is however realized that other shapes of the further post elements 14 may as well be used. Moreover, the further post elements 13 are spaced 100±10% micrometer apart. The further filtering stage is configured to trap any possible large cell debris from entering the core of the cell filter 1. Such large cell debris may intefere with the cell analysis. For symmetry purposes, especially for ease of manufacturing, and to keep the flow homogeneous, the outlet flow channel 3 may also comprise further post elements 14.

Additionally, variations to the disclosed embodiments can be understood and effected by the skilled person in practicing the claimed invention, from a study of the drawings, the disclosure, and the appended claims.

Biological samples, such as for example blood samples, often comprise cancer cells with sizes that overlap with non-cancer cells. This overlapping becomes a challenge for the separation and isolation of cancer cells from non-cancer cells for further analysis. In these samples of interest, cancel cells are rare, i.e., 1 to 10 cells in billions of blood cells, in comparison with the amount of non-cancer cells therein, thus making the separation and isolation of cancer cells even more difficult.

The microscale cell filter of the present invention overcomes the challenge of non-cancer cell contamination and tumor cell heterogeneity, by filtering a whole biological sample by physical characteristics such as size and deformability of the cells contained therein without the need of a pre-treatment to the sample (i.e., filtering, dilution, etc.). This allows to capture, for example, mesenchymal tumor cells that would otherwise be missed by conventional CTC isolation methods. The microscale cell filter is also suitable to process a large volume of sample, up to 10 mL of sample, which is greater than the capacity of other microscale filters known in the prior art. Larger volumes of sample can be processed by fluidically connecting a plurality of microscale cell filters.

In view of separating and isolating cancer cells in a biological sample, white blood cells are often seen as contaminants because their sizes can overlap with those of cancer cells. Some white blood cells are large cells, but usually polynucleated or with a lobulated nucleus, while cancer cells can have large rigid nucleus with an average size well above 5 μm, which make up for around 70% of the cell size.

In this scenario, the presently disclosed microscale cell filter allows non-cancer cells, such as white blood cells, to pass through a plurality of gaps between post elements because white blood cells are flexible and deform along the Z axis, while cancer cells with a size above 5 μm are retained by the gaps between the post elements.

The microscale cell filter is provided with a single row of a plurality of post elements arranged on a substrate between said substrate and a cover, wherein the single row of a plurality of post elements are:
arranged between the inlet flow channel and the outlet flow channel;
arranged perpendicular to the direction of the flow;
the width of each post element is 15 to 40 μm, and
the post elements are arranged parallel to each other.

The size of the post elements allows to provide a plurality of gaps in between the post elements, these gaps are critical for separating and retaining cells according to their size and deformability, while allowing the sample and unwanted cells to naturally flow through the available gaps that are still free for this purpose.

Each gap between the post elements has an aspect ratio between its height and width of 3.5 to 5, this being a static dimension, and even with the post elements being made of different materials, this aspect ratio is maintained. In the context of the present invention, the aspect ratio of the gaps is understood as the ratio between the height that is measured from the substrate to the cover in the region between adjacent post elements and the width of the gap that is measured as the distance between two adjacent post elements.

Another important feature of the microscale cell filter is that the post elements are arranged between the substrate and the cover, wherein the post elements, substrate and/or cover are made of a material suitable to provide wettability and prevent cell adhesion, particularly reducing protein adsorption on the post elements or substrate. This feature allows the cells in the sample to be separated and isolated based on their physical properties without the risk of being retained by adhesion to the surface itself. The microscale cell filter, post elements and/or substrate are manufactured from, comprise, or essentially consist of a material selected from, but not limited to, silicon, glass, paper, and polymers, such as SU-8, PDMS, PC, PET, PE/PET, polyimide, and PMMA, or any combinations thereof. Alternatively, if the chosen material does not provide wettability and prevent cell adhesion by itself, the material may be subject to physical or chemical treatments in order to provide this effect, including oxygen plasma treatments or functionalization with a surfactant.

It is critical for the microscale cell filter to have a single row of post elements arranged parallel to each other, as shown in any of the FIGS. 1 to 8. Besides facilitating the separation and isolation of cancer cells in a cell sample, this single row arrangement facilitates downstream analysis of cancer cells, enabling high resolution imaging after separation of cancer cells because the cells retained in the gaps are well distributed along the row of post elements, and in one focal plane for imaging, making this microscale cell filter particularly suitable for high resolution fluorescence imaging. Other prior art cell filters, such as US2016146778, US20050042766 or Morton, K. L, et al., PNAS, 2008, have multiple rows of post elements, which are not suitable for downstream imaging analysis in situ, or for recovering the separated cells. In these documents, the multiple rows increase unwanted retention of cells, such as white blood cells.

Furthermore, the microscale cell filter of the present invention allows for the flow rate to be adjusted according to the type of cancer cells to separate, substantially reducing the time required to process samples to around an hour. The biological sample must be pumped at sufficient high speed through the microscale cell filter to avoid unwanted blood cells being trapped in the filter. Thus, the flow rate can be adjusted between 80 to 150 μL/min. The selection of this range is not made at random, but it was found to provide the necessary linear velocity of cells against the microscale cell filter. This flow rate range is the most suitable to achieve a linear velocity of the cells that is sufficiently high to allow the large unwanted cells, such as white blood cells, to deform and pass through the gaps between the post elements while still trapping efficiently the cancer cells of interest without compromising their integrity. Linear cell velocities below or above this range in the presented embodiment are not sufficient to achieve the desired effect of separation and isolation of cancer cells, and depletion of non-cancer cells. Consequently, the internal pressure in the microscale cell filter is between 1 and 2 bars. The linear velocity achieved is between 0.5 and 1.5 mm/s.

Furthermore, by using this microscale cell filter, the separated and isolated cancer cells can be recovered for downstream analysis by reversing the flow of the microscale cell filter.

It is important to note that the microscale cell filter cannot be operated by capillary flow, as opposed to other devices in the prior art, such as US2016146778, US20050042766 or Morton, K. L, et al., PNAS, 2008. These devices would not be able to process samples at the desired flow rate or to achieve the desired linear velocity of the cells against the filter. Also, the devices would not be able to process samples of the desired volume (between 1-10 mL), as they would be limited by the inner volume of the device (around 50 uL).

The microscale cell filter has been tested for the successful isolation of viable circulating tumor cells in several cancer types, namely bladder cancer, breast cancer, esophageal cancer, renal cell carcinoma and colorectal cancers, among others, independently on the cell phenotype, and including epithelial, mesenchymal and transitioning cells.

Thus, unlike other devices in the prior art, the microscale cell filter presently disclosed allows to combine enhanced separation efficiency and purity, given that it achieves a cancer cell isolation efficiency up to 80% and white blood cell depletion above 99%, resulting in a purity of round 10%, which is much higher than the 0.05% purity offered by other prior art devices.

The features of the microscale cell filter allow enumeration and downstream analysis of the captured cells, that has been successfully correlated with patient prognosis, such as overall survival, progression-free survival, patient monitoring and stratification, evaluation of resistance to treatment, and analysis of druggable targets.

1. A microscale cell filter for separating and isolating cancer cells within a sample, the cell filter comprising:
an inlet flow channel;
an outlet flow channel;
a substrate and a cover; and
a single row of a plurality of post elements arranged between said substrate and the cover;
wherein the single row of a plurality of post elements are:
arranged between the inlet flow channel and the outlet flow channel;
arranged perpendicular to the direction of the flow;
the post elements are arranged parallel to each other;
wherein the width of each post element is 15 to 40 micrometers;
wherein the plurality of post elements is interspaced, thereby forming a plurality of gaps, each gap being formed in between two adjacent post elements; wherein the single row of a plurality of post elements are arranged such that a flow of the sample flowing from the inlet flow channel to the outlet flow channel passes through the plurality of gaps at a linear velocity between 0.5 and 1.5 mm/s;
wherein the single row of a plurality of post elements are arranged such that a width of each gap is 3 to 8 micrometers; and
wherein each gap has an aspect ratio between its height and width in the range of 3.5 to 5;
wherein the substrate, the cover and the single row of a plurality of post elements are made of a material suitable to provide wettability and prevent cell adhesion or have a treatment suitable to provide wettability and prevent cell adhesion; thereby trapping the cancer cells of the sample at an upstream side of the single row of a plurality of post elements.

2. The microscale cell filter according to claim 1, wherein each of the plurality of post element are cylindrically shaped.

3. The microscale cell filter according to claim 2, wherein the cylindrical shape is a circular cylinder.

4. The microscale cell filter according to claim 1, wherein a height of the single row of a plurality of post elements are identical.

5. The microscale cell filter according to claim 1, wherein the single row of a plurality of post elements is integrally formed with the substrate and the cover.

6. The microscale cell filter according to claim 1, wherein the cover, the substrate and the single row of a plurality of post elements are made of a material selected from the group consisting of silicon, glass, paper, polymers such as SU-8, PDMS, PC, PET, PE/PET, polyimide, PMMA, and combinations thereof.

7. The microscale cell filter according to claim 1, wherein the cover is transparent.

8. The microscale cell filter according to claim 1, wherein a surface of the single row of a plurality of post elements comprise a surfactant.

9. The microscale cell filter according to claim 8, wherein the surfactant is a pluronic acid.
